(19) United States
(12) Patent Application Publication
(10) Pub. No.: US 2012/0010488 A1
(43) Pub. Date: Jan. 12, 2012

(54) Method and apparatus for improving personnel safety and performance using logged and real-time vital sign monitoring

(76) Inventors: Barry J. Henry, Lafayette, LA (US); Chad E. Howell, Maneville, LA (US)

(21) Appl. No.: 13/135,340

(22) Filed: Jul. 1, 2011

Related U.S. Application Data
(60) Provisional application No. 61/360,542, filed on Jul. 1, 2010.

(51) Int. Cl. A61B 5/145; A61B 5/00

(52) U.S. Cl. 600/365; 600/300

(57) ABSTRACT

Group (including, without limitation, occupational work forces) safety and wellness monitoring utilizes baseline physiology testing and vital sign monitoring and sampling technologies. Aerobic capacity of individual members of a group is determined through initial baseline testing which results in an individual health risk assessment. Thereafter non-invasively observed and monitored during incremental work and exercise is commenced. Subsequent data collected is used for advance identification of personnel at risk of injury, such as during unexpected vital sign elevations that can signal early onset of fatigue and heat stress/dehydration prior to injury.

### CROSS REFERENCES TO RELATED APPLICATIONS

None

This application claims the benefit of U.S. Provisional Patent Application Ser. No. 61/360,542, filed Jul. 1, 2010, which is incorporated herein by reference for all purposes.

### FEDERALLY SPONSORED RESEARCH

Not Applicable

### BACKGROUND OF INVENTION

1. Field of Invention

The present invention pertains to a method and apparatus for improving personnel safety and performance including, without limitation, in occupational settings. More particularly, the present invention pertains to a method and apparatus for identifying fatigue in order to prevent injuries and improve overall safety. More particularly still, the present invention pertains to a method and apparatus for real-time detection of the onset of fatigue in order to identify, in advance, specific personnel who are at risk of accidents or other safety-related incidents.

2. Description of Related Art

Occupational safety and wellness programs are widely known and practiced in many different settings. Frequently, such programs are advanced and/or articulated in terms of attitudes or behavior. This concept, often referred to generally as “behavior-based safety”, represents the model upon which most major industrial organizations base their safety and wellness programs. While behavior-based safety programs have reported much success with regard to the attempted prevention of workplace accidents, such programs routinely overlook a critical biotic antecedent to occupational safety incidents—namely, fatigue.

Fatigue drastically degrades employee performance and mental aptitude. Continued activity on the part of personnel suffering muscle fatigue frequently leads to bone, tendon, cartilage, and ligament injuries. In particular, stabilizing muscles (i.e., core muscles) in the hips, shoulders, legs and feet must maintain endurance for more powerful muscles to function properly. Once fatigue occurs, the risk of injury increases exponentially and personal performance is severely degraded.

An overwhelming majority of non-fatal workplace injuries are orthopedic in nature and, therefore, ultimately preventable. Accordingly, a “biology-based safety” program is needed that measures fatigue (and/or unexpected vital sign elevation that signals the onset of fatigue), ideally in real-time, in order to identify those specific individuals who may be at greater risk of injury.

### SUMMARY OF THE INVENTION

The present invention pertains to a method and apparatus for monitoring personnel including, without limitation, detecting the onset of fatigue, in real time or over the course of a work shift, in order to specifically identify personnel who are at increased risk of accidents or other safety-related incidents. The “biology-based” approach of the present invention is not intended to completely replace existing “behavior-based” safety and wellness programs. Rather, the present invention can complement such existing approaches, thereby promoting a safe and efficient workforce.

In the preferred embodiment, the “biology-based” safety system of the present invention initially commences with a needs-analysis in the form of a baseline physiology test. Said baseline physiology test is used to assess an individual's ability to perform strenuous or laborious activity for periods of time indicative to those typical in an industrial work place setting.

Accurately measuring an individual's baseline physiology involves physical effort sufficient in duration and intensity to fully tax the individual's aerobic energy system. In general, clinical and athletic baseline testing typically involves a graded exercise test (either on a treadmill, cycle ergometer, or hand ergometer) in which exercise intensity is progressively increased while ventilation, blood lactate/glucose, vital signs, and oxygen uptake by the body's tissues are measured along with the individual's perception of the exercise effort.

After data is measured and recorded to reflect a unique biological landscape of a particular individual in a group, combined with that individual's medical history, a preferred medical monitoring technology is used to monitor the individual members of a group. Among the dimensions qualitatively measured can include, but are not necessarily limited to, body mass index, blood pressure, height and weight, pulse, VO2 Max and/or VO2 Sub-Max VO2 Threshold, heart-rate max and threshold, skin temperature, body position, respiration, lactate threshold and stress/wattage load during activity and at rest. Over time, individuals can be provided with highly individualized data with regard to their unique physiological and biological conditions; eventually, members of the monitored group can attain biological self awareness that provides value to the individual at work and away from the workplace.

When applicable, data measured through such monitoring can be utilized in connection with a group's preexisting safety and wellness program. To communicate and effectively integrate this data with a group's preexisting health and wellness program, a process known as “Human SCADA” (Supervisory Control and Data Acquisition) can be utilized. Through some desired combination of wireless communication and broadband fiber optic and satellite technologies, users can be provided with a graphical animation of process data. Such data can be beneficially coupled with alarm summary and alarm logging technologies, such that individuals perceived to be working beyond their personal, previously-quantified aerobic limitations are alerted via wirelessly communicated alarm systems.

In the preferred embodiment, such process data is continuously collected over periods of time and during high and low seasons of work. This data is trended such that an individual's particular biological modes of health and well-being can be identified and mapped. This process allows organizational administrators an opportunity to both better assess the efficacy of their safety programs, and also to determine whether certain individuals should reasonably be removed (either temporarily or permanently) from safety-sensitive positions in which they are most likely to suffer a potentially debilitating and costly incident.

### BRIEF DESCRIPTION OF DRAWINGS

The foregoing summary, as well as the following detailed description of the preferred embodiments, is better understood when read in conjunction with the appended drawings. For the purpose of illustrating the invention, the drawings show certain preferred embodiments. It is understood, however, that the invention is not limited to the specific methods and devices disclosed. Further, dimensions, materials and part names are provided for illustration purposes only and not limitation.

FIG. 1 depicts, in schematic form, the data measurement and monitoring system of the present invention.

FIG. 2 depicts, in tabular form, anticipated physiological demands for particular occupations, as determined from published data.

### DETAILED DESCRIPTION OF A PREFERRED EMBODIMENT

While the present invention is described herein with reference to preferred embodiments, it will be understood by those skilled in the art that various changes may be made and equivalents may be substituted for elements thereof without departing from the scope of the invention. In addition, many modifications may be made to the present invention without departing from the scope of such invention. In addition, many modifications may be made to adapt a particular situation or material to the teachings of the invention without departing from the essential scope thereof. Therefore, it is intended that the present invention not be limited to the particular embodiments disclosed as the best mode contemplated for carrying out this invention, but that the invention will include all embodiments (and legal equivalents thereof) falling within the scope of this disclosure.

The system of the present invention can be used in any number of different applications or industries. By way of illustration, but not limitation, biology-based safety® and Human SCADA™ can be efficiently and effectively used in connection with many different situations including, but not limited to, the following industrial applications: land-based operations, marine-based operations, mining operations, loading docks and harbors, ports, subsea operations (divers, for example), high stress health care settings such as emergency rooms and surgical suites, and air transportation/aviation. As used herein, users of the system of the present invention are described primarily as “employees” for ease of reference and consistency. However, it is to be observed that the present invention can be used in connection with virtually any group such as athletics, and such use is not limited solely to occupational or employment scenarios.

In the preferred embodiment, the method of the present invention typically begins with a registration process wherein an automated account is created for a new employee. A standardized template is provided to said new employee, with said template to be populated with responsive information from the employee. Following input of such responsive information, the file template is uploaded to an administrative portal. At such time, a member and login account can be created for said employee.

An optional message can be sent to the employee, either through an automated system or via a manual trigger. Such message can welcome the employee to the portal and provide directions on subsequent steps in the process, including a secure login account to provide the employee with access to the administrator's portal.

The employee will then typically be provided a health history questionnaire. In the preferred embodiment, such questionnaire, and the employee's responses, will be transmitted via the administration portal using the secure login account provided in the welcome message. The information submitted by the employee in response to the health history questionnaire will typically be evaluated by the administrator's medical personnel. Depending on the results of this evaluation, the employee will either progress to the next step of the process, or they will be informed that the administrator's medical staff will be contacting them to review the questionnaire responses in greater detail. If the administrator's medical staff concludes that the employee is not fit for the planned activities, then the employee will not advance. On the other hand, if the administrator's medical staff concludes that the employee is capable of performing the planned activities, then the employee is cleared to advance to the next step in the process.

The employee is then given a baseline physiology test. The test results are then entered into the employee's profile on the administrator's portal. Introduction and education material is made available to each employee via their secure login to the administrator's portal. Additionally, an optional in-person counseling session can be provided by the administrator. Such in-person counseling session can address, in addition other relevant matters, the meaning of baseline data, comparison's of said baseline data to industry averages, and advice and counseling on how to improve.

The employee can then participate in a biology-based safety® orientation program. Such program can include an overview of the system of the present invention, as well as an outline of the safety, health and wellness components of such system. Additionally, the program can further include an overview of the equipment utilized in connection with the present system, as well as trials of such equipment. If desired, testimonials can be provided, and employee questions can be addressed.

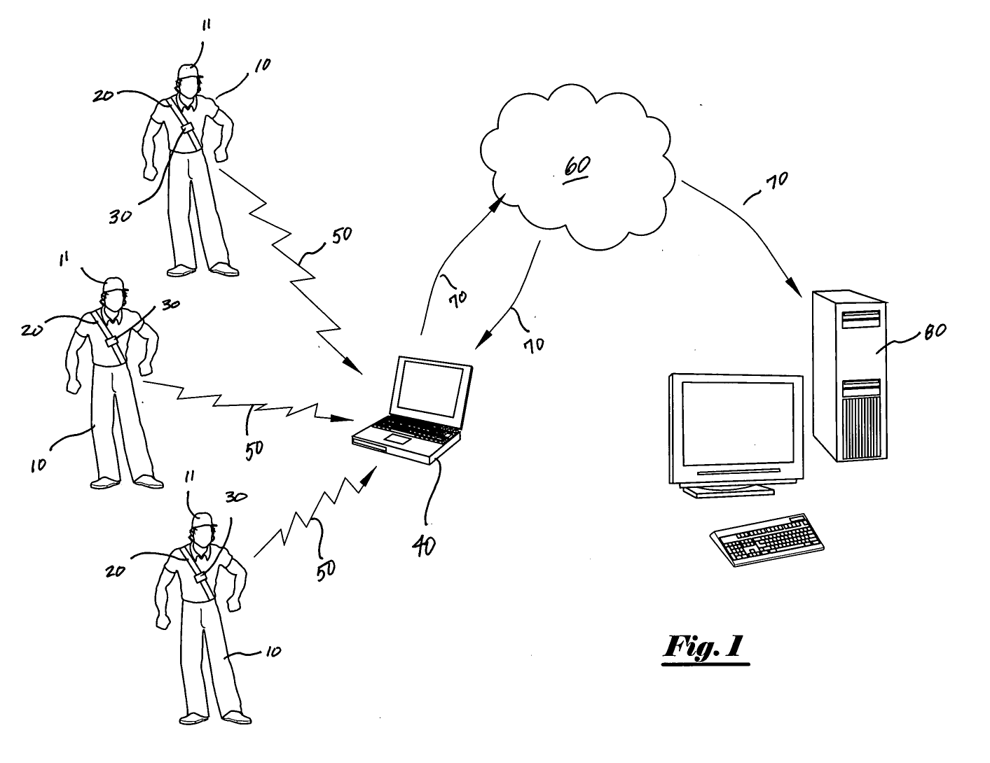

Referring to FIG. 1, following completion of the baseline test and orientation program, employee workers 10 are fitted with worn devices 20 that each contain at least one bio-sensor 30. Each worn device 20 is fitted until comfortable to the employee; in most cases, comfort is crucial for long-term employee commitment to the program. In the preferred embodiment, each worn device 20 should fit snuggly with at least one bio-sensor 30 against each employee's chest wall. If necessary, a shoulder strap can be used to prevent such worn devices from slipping down towards the employee's waist. It is to be observed that the worn devices 20 should beneficially fit each employee 10 correctly in order to ensure accurate data transmission and collection.

Wearing instructions are provided to each employee 10. Specifically, logistics of the daily fitting and wearing of bio-sensor device 30 are explained to the employee. Frequently, protocols and procedures for use of said bio-sensor devices 30 are established by the employer and communicated to the employee 10. Such protocols and procedures can include charging, conduction, signal transmission and troubleshooting. Additionally, sensor device maintenance instructions can include washing and cleanliness of worn devices 20, responsibility for upkeep and cleanliness of bio-sensors 30.

Any concerns or questions regarding the fitting, wearing, and upkeep of the worn devices and/or bio-sensors are addressed. If desired, a representative of the administrator can be assigned to manage the equipment and relationship with the employer (if, as is frequently the case, the employer is not acting as the system administrator). Documentation, including Frequently Asked Questions (“FAQ's”) can be provided to the employee (in both hard copy, as well as on the administrator's portal).

Information can be compiled and analyzed by the administrator to develop a health risk assessment. In many instances, such health risk assessment can be provided to group leaders or employers in the form of an executive level summary. More specifically, data collected from all employee baseline tests can be “scrubbed” and presented in a HIPAA-compliant manner. Such test data can be placed in a health risk assessment report that evaluates particular employee's risks for potential health, safety and/or wellness issues.

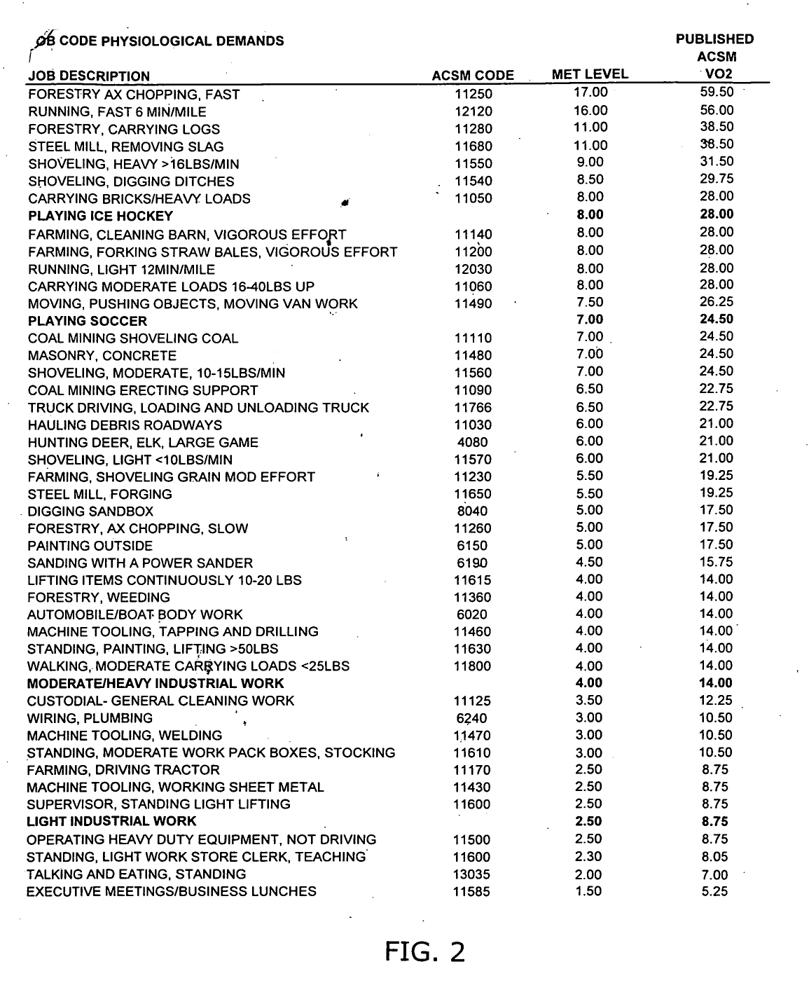

Such health risk assessment is based on a number of variables including traditional medical laboratory values including, without limitation, body-mass index (“BMI”), blood pressure, psychological work aptitude scoring, and other fitness/health parameters. Group leaders are provided with a “snapshot” of their employee population which includes identification of “at risk” members. Frequently, such health risk assessment determines whether vital sign monitoring will be used and, if so, the manner in which it will be deployed. FIG. 2 depicts, in tabular form, anticipated physiological demands for particular occupations, as determined from published, publicly available data.

At this stage in the process, Human SCADA data measurement and monitoring process is typically implemented. Such process can include an in-field evaluation, which can be utilized to evaluate employee “fitness for duty”. Logged vital sign data is collected at all times when an employee is wearing a bio-sensor device 30. In the preferred embodiment, logged Human SCADA™ is performed for a predetermined period of time.

Referring to FIG. 1, measured data can be stored on bio-sensor device 30 being worn, and must be uploaded periodically. Frequency of upload is typically determined by the storage capacity of the particular monitoring technology being used. Once such data is uploaded to a computer 40, the administrator can transmit that data to a main administration server database, typically over the Internet using secured web services. Such in-field collected data is viewed, together with any previously generated health risk assessment, to evaluate the client's status and determine whether an employee is “fit for duty”. Further, the in-field evaluation provides an objective overview of workforce fitness in a specific work environment.

Such Human SCADA can typically be divided into two separate groups: long-term logged Human SCADA, and real-time Human SCADA. Long-term logged Human SCADA provides for data trending, audit coverage for “fitness for duty” assessments, biology-based safety certifications, fatigue prevention during long work shift cycles (such as, for example, 14 days on/off cycles of work common in many industrial settings) development of objective education curriculums from the trended data to teach or reinforce new and appropriate behaviors in the workplace, and use within workplace health and wellness programs along with other similar applications.

Real-time Human SCADA™ typically comprises real-time monitoring of vital signs and other biological or physiological data, deployed in specific applications determined by a program administrator and an employer. During real-time vital sign monitoring, data is transmitted from the monitoring device to a local receiver (such as computer 40) via wireless technology including, but not limited to, Bluetooth, Wi-Fi, or radio. Such real-time measured data can be reviewed on site at a local computer 40 or remotely using the administration portal. Even while real-time monitoring is taking place, data can still be logged locally on a monitoring device and can be uploaded to the server as described above.

Furthermore, local notification through signaling devices such as but not limited to auditory, visual color changes via lighting devices and/or displays, vibratory devices, and such other methods of notification can readily assist workers and supervisors to avoid potentially hazardous situations. When appropriate, such signaling devices can be connected to hard hats (such as hard-hats 11), work boots, work gloves, uniforms, or other items worn by employees 10. Such signaling devices can be connected to items that must be worn by a particular employee to reduce the likelihood that the employee will choose not to wear such signaling device.

In a preferred embodiment, signaling can be beneficially achieved through reflex arc software programming that automates a local signal response when pre-programmed physiologic thresholds or levels are exceeded and detected by a worn bio-sensor. The response can occur locally without need for off-site processing because the data exceeds critical levels and demands an immediate safety stop in the workplace to avoid worker/workplace injury. Pre-programmed thresholds or levels are unique to an individual based on health and baseline/updated physiologic testing data measurements derived from this innovative program of occupational safety technology.

Vital sign data can also be paired with environmental data such as temperature, climate, oxygen levels, noxious gas level, micro-environment of personal protective equipment, and other weather/environmental data to make the safety data more robust for on-site reflex signals and off-site command center processing.

Additionally, there are an increasing number of individuals with chronic illness working in industrial workplaces with aggressive environmental conditions. Such individuals can pose a risk to themselves and others if their condition such as diabetes or heart disease worsens while on the job. Collecting data from implanted devices such as glucose monitors and pacemakers/defibrillators and other such devices wirelessly to pair with vital sign and local environmental conditions through software programs that are designed to evaluate real-time and over the course of a work shift significant changes pertinent to fatigue and worsening chronic conditions are invaluable methods to improve individual and work-site safety.

The problem of sleep disorders and fatigue related decision making errors is well documented in many industrial setting such as transportation, crane/heavy equipment operation, aviation, and like industrial settings. The method of gathering sleep information and data points is currently expanding to affordable and easily deployed device technology. The method of collecting and pairing sleep data with real-time and logged vital sign data provides an additional layer of data analysis for predicting and preventing fatigue related incidents and injuries. Therefore, the comprehensive program of collecting multiple unique human data points and providing a green, yellow, red scale of safety monitoring on-site and off-site via wireless, cloud based, local and mainframe server technology paired with a command center for individuals performing work real-time and over a work shift is the proposed method of industrial safety non-existent prior to this innovative program described herein.

In the preferred embodiment, specialized integration software is used to collect all monitored data and transmit such data, typically via the Internet, to the administrator's server. Such integration software typically comprises two components: a client application and an Internet-based web services suite.

A software application can be tailored to run in the background an employer's computer environment, and can ideally access local database(s) containing data stored on third-party software. Said application extracts data collected from employees, including data stored on such third-party software. The data is then packaged into an electronic message and pushed to the administrator's server, via web services, over the Internet.

The Internet-based web services portion of the integration software can be hosted on the administrator's web server. In a preferred embodiment, no front end exists for this application; instead a set of securely exposed web services accept specific electric messages containing member data from the field. Once a message is received, the logic within this application parses all data from the electronic message and drops such data into the administrator's database.

The connection between the employer application and the Internet-based web services is secured. Such security is provided using “HTTPS” and username token authentication. In addition, all data is prepackaged as an encrypted data object before being transmitted. The access code to decrypt this data exists only on the administrator's dedicated server, which is located behind a hardware firewall. Upon delivery, the administrator's web service analyzes the integrity of the data object to ensure that the message has not been intercepted, hacked or tampered with and finally authorizes the delivery and processing of the data.

The administration portal is an interface through which participating employees and the administrator can communicate. Through this internet-based environment, the administrator and participating employees can engage in various activities including, but limited to, counseling, education, discussion, trending, reporting, and analysis.

In the preferred embodiment, the administration portal will consist of, but not limited to, the following role-based environments:
Employee Environment—A secure portal account will be provided for all employees participating in the administrator's service package. Login credentials will be provided to each member during the registration process, which will give each employee access to various information including, but not limited to, their personal baseline test data, daily monitoring reports, safety education, and health/wellness coaching.
Employer Management Environment—Group leaders will have the ability with an advanced role to view high-level reports of their workforce risk and fatigue levels, without any access to individual identities of employees or sensitive information.
Real-Time Environment—following integration of real-time data, the system transfers monitored data from the field to the administrator's database. The administration portal can then retrieve such data to display employee vital signs with sub-second delay from data collection.
Administration—an administrative function can be provided to a controlled group within the system administration. Employee data can be viewed and modified. Access to different sections of the administration portal can be controlled.
Education, Training, and Collaboration Community—an environment for employees to communicate and collaborate with administration, as well as other employees involved in the program. Forum and discussion boards are available for question and answer as well as discussion of health challenges and motivation. Reminders and alerts are provided to members for upcoming certification renewals and other communication.

Certain areas of the administration portal are secured. Such secured areas use a “HTTPS SSL” certificate. Further, every employee having access to the administration portal has a unique username/password account, which is used to authenticate valid and active users.

Employees that operate heavy or potentially hazardous equipment, devices, or machinery, work in strenuous or stressful environments or have professions which they are responsible for their coworkers, passengers, or others wellbeing may be subject to a pre-work questionnaire. In the preferred embodiment, the questionnaire will be delivered electronically to the individual and will be required to be completed prior to working or taking control of a device or equipment.

Such questionnaire can include, without limitation, the following Yes or No Questions
Do you feel Fatigued?
Have you consumed alcohol in the last 24 hours?
Have you taken any narcotics in the last 24 hours?
Do you have any pain or discomfort anywhere?
Do you feel dizzy or light headed?
Have you participated in any rigorous physical activity?
Have you experienced an injury recently?
Did you sleep well last night?
Do you sleep well generally?

Referring back to the drawings, FIG. 1 is a schematic depiction of a representative deployment of the method for monitoring personnel workplace safety of the present invention. At least one biometric sensor 30, deployed on worn device 20 (which can be a harness, vest or other item), is placed on at least one worker 10. Physiological data of said at least one worker 10 is measured using said at least one sensor, wherein said measured physiological data can include, without limitation, skin temperature, VO2 Max and/or VO2 Sub-Max VO2 Threshold, body position, heart-rate or respiration. A glucose/insulin monitoring device can also be employed to measure blood levels of glucose, insulin dosing, lactate, or hemoglobin/hematocrit values for workers 10. Local environmental conditions such as temperature, climate, noxious gases, hypoxic air, and micro-environment of personal protective equipment can also be measured at the same time, using biometric sensor 30 and/or other capable sensor(s).

Such measured data can then be transferred, ideally via wireless transmission 50, to a local computer 40 having desired software. Such measured data can then be transferred from said local computer 40 over the Internet (70) to a dedicated server, such as cloud server 60. Such data can, in turn, be transferred to a remote computer 80, again over the Internet (70). Although remote computer 80 can be located in any number of different places, said computer 80 is located at a command center operated by a system administrator; said system administrator who administers the method of the present invention.

When measured values reach certain predetermined levels, a signal can be provided to workers 10. Such signal can be a visual, auditory, or vibratory signal, or combination thereof. Moreover, said signal can be on hard hat 11, or other beneficial location.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention. The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. A method for monitoring personnel safety comprising the steps of:
a. placing at least one biometric sensor on a person;
b. measuring physiological data of said person using said at least one sensor;
c. transferring said measured physiological data to a first computer having software; and
d. generating a signal when said measured physiological data meets at least one predetermined value.

2. The method of claim 1, further comprising the step of performing a medical examination before placing said at least one sensor on said person.

3. The method of claim 1, further comprising the step of transferring said data from said first computer over the Internet to a second computer.

4. The method of claim 1, wherein said physiological data comprises at least one of the following: skin temperature, VO2 Max and/or VO2 Sub-Max VO2 Threshold, body position, heart-rate or respiration.

5. The method of claim 3, further comprising the step of storing said measured physiological data to a dedicated cloud server.

6. The method of claim 5, wherein said dedicated cloud server stores collected physiological data from a plurality of locations.

7. The method of claim 3, wherein said data is transferred using a web service application providing at least two points of access.

8. The method of claim 5, further comprising a data transaction service for allowing a software integration module to transfer data from an active location to a database.

9. The method of claim 5, further comprising a data query service for allowing a software integration module to transfer data from the cloud server to said second computer.

10. The method of claim 5, wherein said server hosts web applications.

11. The method of claim 10, wherein said web applications comprise dashboards, control panels or business modules.

12. A method for monitoring personnel workplace safety comprising the steps of:
a. placing at least one biometric sensor on a worker;
b. measuring physiological data of said worker using said at least one sensor, wherein said measured physiological data comprises skin temperature, VO2 Max and/or VO2 Sub-Max VO2 Threshold, body position, heart-rate or respiration;
c. transferring said measured physiological data to a first computer having software; and
d. generating a signal when said measured physiological data meets at least one predetermined value.

13. The method of claim 12, further comprising the step of transferring said data from said first computer over the Internet to a second computer.

14. The method of claim 13, further comprising the step of storing said measured physiological data to a dedicated cloud server.

15. The method of claim 12, wherein said signal comprises a visual, auditory, or vibratory signal.

16. The method of claim 12, further comprising the step of measuring local environmental conditions.

17. The method of claim 16, wherein said local environmental conditions comprise temperature, climate, noxious gases, hypoxic air, and micro-environment of personal protective equipment.

18. The method of claim 12, wherein said sensor comprises a glucose/insulin monitoring device.

19. The method of claim 19, wherein said measured data comprises blood levels of glucose, insulin dosing, lactate, or hemoglobin/hematocrit values.
